(19) United States
(12) Patent Application Publication
(10) Pub. No.: US 2024/0181201 A1
(43) Pub. Date: Jun. 6, 2024

(54) METHODS AND DEVICES FOR HEARING TRAINING

(71) Applicant: EARGYM LTD, London (GB)

(72) Inventors: Amanda PHILPOTT, London (GB); Andrew SHANKS, London (GB)

(73) Assignee: EARGYM LTD, London (GB)

(21) Appl. No.: 18/556,257

(22) PCT Filed: Apr. 27, 2022
(86) PCT No.: PCT/EP2022/061259

§ 371 Date: Oct. 19, 2023

(30) Foreign Application Priority Data
Apr. 29, 2021 (EP) 21171347.4

(51) Int. Cl. A61M 21/00

(52) U.S. Cl. CPC A61M 21/00; A61M 2021/0027; A61M 2021/005; A61M 2205/502

(57) ABSTRACT

A computer implemented method, user device and non-transitory computer-readable medium storing instructions for performing hearing training with a user device comprising a user interface and an audio output, the training comprising: providing a background audio signal and a target audio signal using the audio output, the target audio signal at least partially overlapping with the background audio signal, and the target audio signal defining information to be determined by a user; wherein one or both of the background audio signal and target audio signal comprise binaural audio; receiving, at the user interface, a user input corresponding to a user assessment of the information defined by the target audio signal; and providing feedback to the user based on the user assessment indicated by the user input.

### FIELD

The invention relates to computer implemented methods for performing hearing training, in particular methods using smart user devices. In particular the invention provides a particularly effective and convenient means of training the hearing of users.

### BACKGROUND

Humans hear or perceive sounds by detecting vibrations in the air through their ears. The sense of hearing is a key manner by which humans interact with their environment.

However, hearing loss is a common affliction for many people. Age-related hearing loss can occur gradually over time and is particularly common in those over the age of 60. Equally noise-related hearing loss can occur when people are exposed to loud noises such as machinery, explosions or gunfire and loud music.

It will be appreciated that people suffering from hearing loss experience both diminished sensory response to sounds, and increased cognitive load as their brains struggle to adapt to their change in hearing and have to work harder to process and discern noises. As such, a training method which can help a user to better appreciate sounds and/or reduce the cognitive load associated with hearing is highly desirable. Indeed, there is a particular lack of tools available to help those with age-related or noise-related hearing loss.

Methods for measuring, monitoring and training hearing have previously been suggested. However, these approaches typically suffer from a number of common issues.

Most critically, existing approaches fail to accurately reflect real life. The tasks and techniques involved offer poor training to users in their daily lives. Furthermore, tests and training often based in a laboratory and requires specialist equipment. As such, these approaches can be inaccessible to many users. Finally, whilst different users may have significantly different ranges or levels of hearing loss, traditional methods often involve regimes which are the same for each user. This inflexibility reduces effectiveness of these existing approaches.

Therefore, there is a clear need for improved hearing training methods, systems and devices which overcome at least some of the issues identified above.

### SUMMARY

In accordance with an aspect of the invention there is provided a computer implemented method of performing hearing training with a user device comprising a user interface and an audio output, the method comprising: providing a background audio signal and a target audio signal using the audio output, the target audio signal at least partially overlapping with the background audio signal, and the target audio signal defining information to be determined by a user; wherein one or both of the background audio signal and target audio signal comprise binaural audio; receiving, at the user interface, a user input corresponding to a user assessment of the information defined by the target audio signal; and providing feedback to the user based on the user assessment indicated by the user input

It will be appreciated that this aspect of the invention provides a particularly realistic and therefore effective method of training the hearing of a user. The training imitates the sounds and situations a user experiences in their day-to-day lives.

A user must distinguish the target audio signal from the background audio signal, determine or identify the information conferred by the target audio and provide a user input that relates to their understanding of the target audio. On receiving feedback the user may appreciate whether their assessment of the target audio signal was correct, and as such may develop their hearing skills. The hearing skills which may be developed through this method include: sound detection; localisation; discrimination; intelligibility in quiet; and intelligibility in noise.

The invention utilises binaural audio, by this we mean audio comprising two different audio channels (i.e. right and left audio channels) each configured to independently provide the same sound to a respective ear of a listener, wherein the audio channels differ based on an assumed arrangement of the ears relative to one another. For instance, the binaural audio discussed above may be recorded using two microphones positioned in a similar manner to human ears on either side of a model head or human head. This approach is often referred to as “binaural recording” or the process of binaurally recording sound. The difference between the sound recorded by each microphone—i.e. the differences between the two audio channels of the binaural audio—is defined by the relative positioning of the two microphones on the model or human head which ideally approximates the relative position of the ears of a listener. Alternatively, the binaural audio discussed above may be synthesised or generated artificially from conventional audio, by forming the two audio channels from the conventional audio using a transfer function (i.e. a head-related transfer function (HRTF)) that defines an assumed relationship between a user's ears. In particular, the difference between the right and left audio channels of binaural audio may be based on a head-related transfer function (HRTF) which characterises how each ear receives sound from a specific point in space. In preferred examples the binaural audio may be adapted or varied dependent on the relative positioning and orientation between the head of a user and the apparent source of sound(s) within the binaural audio. A variety of software products are available to perform the generation of binaural audio from monophonic or stereophonic sound, including the ‘AMBEO Orbit’ plug-in produced by Sennheiser Electronic Gmbh & Co. and ‘DearVR MICRO’ plug-in produced by Dear Reality GmbH which are both plug-ins for the ‘Pro Tools’ audio production software produced by Avid Technology, Inc.

In summary, binaural audio is specific example of stereophonic audio (also referred as “stereo audio” or “stereo”) where the difference between right and left audio channels is based on an assumed relationship between the listeners' ears. This assumed relation corresponds to a head-related transfer function (HRTF) for each ear, which is not present in conventional stereophonic audio.

As discussed above, the binaural audio within the target audio signal and/or background audio signal may be binaurally recorded and/or may be generated or synthesised from a respective input audio signal (e.g. a respective monophonic or stereophonic input audio signal).

The inventors have recognised that the use of binaural audio in hearing training is particularly beneficial. In contrast to conventional monophonic audio (also referred to as “monaural audio”, “mono audio”, or “mono”) or stereophonic audio, binaural audio is particularly similar to the sounds heard in real life by a user. This allows for training to imitate situations and tasks commonly experienced by users particularly accurately. Furthermore, binaural audio allows a user to train their spatial resolution of sounds—i.e. the ability or skill of a user to identify the location or source of sounds based on the difference in how sound is heard by each ear. This is a skill that is frequently used by healthy humans but is particularly difficult for those with hearing loss, especially where hearing loss is different in a person's two ears. Such training of a user's spatial resolution of sounds is particularly difficult to achieve using traditional mono or stereo sounds.

Furthermore, the use of binaural audio allows for spatialization. Spatialization is the process of modifying an audio signal to make it localisable for a listener, so that the sound defined by the audio signal appears to originate from a specific location. Spatialized audio allows for the creation particularly complex hearing training situations. Therefore, all binaural audio discussed herein may be spatialized audio, and references to binaural audio may be replaced with spatialized audio where appropriate.

In preferred examples the method comprises tracking the position and orientation of the head of the user relative to an apparent source of a sound within the binaural audio of the target audio signal and/or background audio signal. Subsequently the method may comprise adapting the binaural audio in the target audio signal and/or background audio signal based on the position and/or orientation of the head of the user relative to the apparent source of the sound, such that the location of the apparent source appears consistent to the user. Thus the method can involve so-called “head tracking”. The head tracking may be performed using cameras configured to detect movement of the head of the user, accelerometers or sensors mounted to the head of the user or the user device and/or any other suitable method. Alternatively the position and orientation of the head of a user may be determined or assumed based on the position and/or orientation of the user device. The binaural audio produced through these steps is sometimes referred to as ‘reactive binaural audio’ or ‘adaptive binaural audio with head tracking’. Such binaural audio may be generated from conventional mono or audio signals using a head-related transfer functions (HRTFs) that depend on the position and orientation of the listener. Suitable tools to achieve this include the plug-ins discussed above.

The use of binaural audio that depends on the position and orientation of a user relative to an apparent source of a sound is particularly realistic. Hence, the hearing training is particularly effective at improving the hearing skills used by a user in their day-to-day life.

Spatialized audio provides particular benefit when combined with virtual reality (VR) settings and training environments as will be discussed further below.

The target audio signal and background audio signal at least partially overlap. By this it will be understood that the signals are provided simultaneously or synchronously to the user, such that at least some sounds within each signal are heard together by the user. Users with hearing loss may find it difficult to distinguish between such complex arrangements of overlapping audio signals. Such overlapping audio signals accurately reflect real life. Consequently, hearing training in accordance with this aspect of the invention is particularly effective. In particular, the background audio signal may act as a distraction for the user, requiring them to work harder to discern and interpret the target audio signal within the “noise” of the background audio.

Preferably the background audio signal comprises binaural audio and wherein the background audio signal defines two or more sounds which have different apparent sources relative to the user. The use of a background audio signal with multiple sounds arranged at different apparent locations is particularly realistic. Additionally or alternatively, the background audio signal and target audio signal each comprise binaural audio and wherein the background audio signal defines one or more sounds which each have a different apparent source relative to the user than a sound defined by the target audio signal. By providing a plurality of sounds positioned at different apparent sources it is possible to generate a particularly realistic “soundscape” (analogous to a landscape) which corresponds to situations a user may encounter in their day-to-day life. Thus the hearing training is particularly effective at improving the hearing of a user in normal situations. The binaural audio of each sound can be adjusted based on the position and orientation of a user relative to the apparent source(s) in the manner discussed above.

By providing feedback to a user based on the result of the user assessment the user may appreciate whether they correctly identified the information conferred by the target audio signal. As such, the user is guided to improve their hearing skills. For instance, the method may comprise providing feedback to the user in the form of an audio indication, visual indication and/or tactile indication (e.g. vibration of the user device) to indicate whether they have correctly understood the information defined within the target audio signal. In further examples the feedback may also be partially based on the time required for the user to assess the target audio signal and provide a user input.

As discussed above, the target audio signal defines information to be determined by a user. The target audio signal may be configured to convey or confer information in any suitable manner. For example the information defined by the target audio signal may comprise: a target location within a training environment;

the content of the target audio signal and preferably the linguistic content of speech within the target audio signal; and/or, similarity and/or relationship to a second target audio signal.

As such, the target audio signal may provide a user with information directly through the content of the signal—e.g. where the information is defined by specific sounds and/or words within the target audio signal. Alternatively the target audio signal may indirectly confer information to a user, in which case the user may be required to interpret the target audio signal to identify the information—e.g. a location to be identified, and/or similarity or relationships to other sounds.

The actions or tasks required from the user during the hearing training may take different forms depending on the manner in which information is conveyed to the user by the target audio signal.

For example, where the information defined by the target audio signal comprises a target location within a training environment, providing the target audio signal may comprise: receiving, at the user interface, one or more preliminary user inputs each corresponding to an intermediate location within the training environment; and varying one or more properties of the target audio signal based on the relative positions of the intermediate location and the target location within the training environment.

Depending on the distance or other relationship between the target location and the intermediate location, the sounds heard by the user will change. Therefore, a user may identify the target location based on the changes in the target audio signal and/or background audio signal as the intermediate locations they input changes. As such, the method may comprise comparing the intermediate location(s) input by a user to the target location and varying the audio signals provided to the user based on the result of this comparison. Preferably this comparison is performed by the user device, although this is not essential and may instead be performed by a separate device or system. Having identified what they believe is the target location, the user may provide a user input corresponding to their assessment of the target location. As such, the user input received during the method may be an indication of a location the user believes corresponds to the target location based on the variations in the target audio signal. A user may be understood to have correctly identified the position of the target location if their user input corresponds to the target location and/or is within a predetermined distance of the target location.

Thus in this training method a user is seeking or foraging for a target location within the training environment which may be visually hidden from the user based on the audio signals provided. This may develop the sound detection, discrimination and localisation skills of the user. The training environment may be a physical environment in which the user is located, but more preferably is a training environment displayed to the user (as will be discussed further below).

These seeking or foraging training methods are designed to improve the sound detection hearing skill of a user. Cognitively a user may improve their attention span and spatial working memory. As such, these types of methods are designed to help users stay safe during their daily lives and improve their abilities with spatial activities and tasks such as sports.

Varying one or more properties of the target audio signal may comprise one or more of the following: varying the volume of the target audio signal relative to the background audio signal; varying the content of the target audio signal; varying the pitch, duration, reverb and/or rhythm of the target audio signal; or, where the target audio signal is binaural, varying the apparent source of the target audio signal relative to the user. In this final example, the apparent source of the target audio signal may be “panned” relative to the user as the intermediate location is varied. In such cases a user may be required to identify a target location within the training environment at which the apparent source of the target audio signal appears to be emitted from a source positioned directly in front of the user. For example, the apparent source of the target audio signal may initially be located to the right or left of the user, behind the user and/or a long distance from the user. Based on the intermediate inputs the apparent source may be moved relative to the user, and the user may be required to attempt to identify the location at which the apparent source of the target audio signal is positioned in front of them and/or close to them. Varying the target audio signal may be performed by the user device (e.g. by a processor comprised within the user device) or an external device or system (e.g. a remote or cloud-based system in communication with the user device)

The target location may be static or may change position within the training environment. In particular, changing the position of the target location periodically or continuously over time can increase the difficulty of the hearing training.

In further preferred examples, the information defined by the target audio signal may correspond to a target visual component within a plurality of different visual components within a training environment. Thus the user may be required to “identify” or select an appropriate visual component from within the training environment in response to hearing the target audio. Thus the method may comprise receiving a user input corresponding to a visual component the user believes relates to the content of the target audio signal.

In preferred examples the visual component may be a button or displayed object within the training environment. For example, the training environment may comprise a menu within a café, bar or restaurant and a plurality of visual components corresponding to menu items served by the café, bar or restaurant. In response to hearing a target audio signal containing a customer order the user may be required to select one or more menu items using the user interface the customer desires. Alternatively, the training environment may comprise a plurality of animals forming the visual components, whilst the target audio signal may comprise an animal call. In response to hearing the animal call as part of the target audio signal the user may need to select the appropriate animal via the user interface.

Therefore in such training methods the user is tasked with identifying a correct visual component based on the content of the target audio signal. This identification task requires the user to distinguish the target audio signal from the background audio signal and select the appropriate visual component corresponding to the information within the target audio signal. This method helps train the intelligibility in noise skill of a user as well as their working memory and attention.

These types of identification tasks are designed to help develop the intelligibility in noise hearing skill of a user. Cognitively, a user may develop their selective attention and focus, improving their ability to focus on a specific object or sound. In particular users may find these types of methods improve their social interactions, especially in crowded or noisy settings.

In further examples, the training method may involve the user “matching” two separate target audio signals. The user may be required to assess whether two target audio signals sound similar and/or are conceptually related. In preferred examples the method may comprise sequentially providing the target audio signal and a second target audio signal to a user using the audio output and receiving a user input that corresponds to a user assessment of whether the target audio signal and second target audio signal are similar and/or related. Such methods can develop the memory of a user as well as their ability to distinguish sounds from one another.

In such examples the method may comprise providing two or more target audio signals using the audio output; and, wherein the user input indicates whether the user believes said two or more target audio signals are similar and/or related. In particularly preferred examples each of the two or more target audio signals may be provided in response to receiving a preliminary user input corresponding to said target audio signals. For instance, in a “tile-matching” training method the user may provide a preliminary input relating to a tile or other selectable visual component and in response a corresponding target audio signal may be provided to a user. Where multiple tiles share similar (e.g. the same) target audio signals and/or related target audio signals, the user may provide a user input indicating that they understand the corresponding tiles match.

These types of matching tasks are designed to help develop the discrimination hearing skill of a user. Cognitively the short term or working memory of a user could be improved, including both the auditory working memory of a user and their visuospatial short term memory (especially in the ‘tile-matching’ examples discussed above). As such, these types of approaches are designed to offer benefits to a user's reading skills, focus and ability to learn languages.

Therefore, as discussed above, the method can involve a variety of different tasks such as foraging/seeking, content identification and matching. In preferred examples, these different tasks form alternative modes of the method of performing hearing training. For instance, method may involve performing one or more of a foraging/seeking mode, an identification mode and/or a matching mode, each of which may take the forms discussed above. In such examples, the method may comprise performing one or more of said training modes based on an input from the user and/or based on the results of a standardised hearing test performed by the user in preliminary method step. Thus it will be appreciated that as the mode of hearing training changes the task required of the user and/or the type or form of the information defined by the target audio signal is changed.

The standardised hearing test may comprise one or more of: the Amsterdam inventory for Auditory Disability and Handicap (AIADH) (‘Factors in subjective hearing disability’ Kramer, Kapteyn, Festen, & Tobi, Audiology, November-December 1995; 34(6):311-20) a series of multiple choice questions in which a user rates how their quality of life is affected by their hearing; HearWHO (World Health Organization, 2018) where users are asked to listen to and identify digits spoken over background white noise or any other speech-in-noise test; a test of the highest frequency a listener can hear; a pure tone audiometry test that tests the threshold volume at which a listener can hear sounds across a range of frequencies (such as the approach defined in ISO 8253-1:2010, published by ISO, 2010-11); or any other suitable test of the hearing skills of a user.

In preferred embodiments, the method further comprises analysing the user input received at the user interface to determine whether the user assessment indicated by the user input corresponds to the information defined by the target audio signal. As such, the method may involve determining whether a user has correctly identified the information conveyed by the target audio signal. Preferably the feedback provided to the user is based on the result of this analysis step. This analysis may be performed by the user device (e.g. by a processor within the user device) or by a further device or system external from the user device (e.g. a remote or cloud-based system in communication with the user device).

Preferably the method involves iteratively repeating the method steps discussed herein. As such, the user can perform the training repeatedly to improve their hearing skills. Thus the user may perform the hearing training multiple times within a training session, the target audio signal (and preferably the information to be determined) and/or the background audio signal being varied between each iteration of the method steps. As such, in preferred examples the method involves iteratively repeating the method steps with a single user—i.e. such that the user inputs received in different iterations are received from the same user.

In preferred examples the method may involve iteratively repeating the method steps for a predetermined period of time (e.g. for a period of time from 3 minutes to 30 minutes), for a predetermined number of iterations (e.g for 10, 20, 30 or 50 iterations), or until the user fails to provide a user input correctly corresponding to the information defined by the target audio signal. Where the method is iteratively repeated a predetermined number of times, the predetermined number may be in the range of 5 to 50, more preferably in the range of 10 to 30.

In such examples, the method may further comprise providing session feedback to a user based on their performance across multiple iterations of the method for hearing training. For example, the session feedback might include an indication of: the total number of iterations in which a user provided a user input that correctly corresponded to the information defined in the target audio signal were received; the proportion of iterations in which correct user inputs were received; and/or the highest number of consecutive iterations in which correct user inputs were received by the device (e.g. the highest streak of correct answers provided by the user). The session feedback may comprise an audio indication, visual indication and/or tactile indication (e.g. vibration of the user device) or any of the other features of the feedback provided after each iteration discussed above. The session feedback may be generated by the user device (e.g. a processor within the user device) and/or by any other device or system (e.g. a remote or cloud-based system in communication with the user device).

More preferably still, based on determining that the user assessment indicated by the user input corresponds to the information defined by the target audio signal, a difficulty of the hearing training may be increased for subsequent iterations of the method. As such this step may be based on the results of the analysing process discussed above. In this manner, difficulty of the hearing training can be increased when a user successfully identifies the information conveyed by the target audio signal. As such, the hearing skills of a user may be further developed through more difficult training as their hearing improves. Additionally, or alternatively, based on determining that the user assessment indicated by the user input does not correspond to the information defined by the target audio signal, a difficulty of the hearing training may be decreased for subsequent iterations of the method. Varying the difficulty of the hearing training allows the training to be personalised to a user, improving training outcomes for individuals.

In other words, the performance of a single user across different iterations of the hearing training may be used to adapt the difficulty of future training. The adaptive difficulty maintains user engagement and continues to challenge and develop the hearing skills of a user over time.

The difficulty of subsequent iterations of the method may be varied in response to each success and/or failure of the user. Alternatively, the difficulty may be varied periodically in response to a user meeting a threshold of successful or unsuccessful user inputs across a number of sequential iterations of the method—e.g. a predetermined number of successes and/or failures in a row, a predetermined proportion of successes and/or failures across a series of sequential iterations of the method. It will be understood that a “success” refers to an iteration in which the user input received from the user correctly corresponds to the information defined by the target audio signal (i.e. that the user has correctly assessed or identified the information within the target audio signal). In contrast in a “failure” the user input will not correspond to the information defined by the target audio signal. Thus the difficulty can be automatically adjusted to meet the needs of the user.

The method may comprise: determining, across a plurality of sequential iterations of the method, the proportion of user assessments indicated by the user inputs that correctly correspond to the information defined by the respective target audio signals; and wherein if the proportion of correct user inputs is greater than a predetermined first value the difficulty of the hearing training is increased for one or more subsequent iterations, or if the proportion of correct user inputs is less than a predetermined second value the difficulty of the hearing training is decreased for one or more subsequent iterations. Therefore, by comparing the proportion of successes and failures of a user to predetermined thresholds the difficulty may be automatically adapted to reflect the performance of the user.

In particular, the inventors have recognised that engagement of a user is significantly increased when the user correctly assesses the information within the target audio signals in approximately 85% of iterations (i.e. the success rate is approximately 85% and the failure rate is approximately 15%). If the user is incorrect significantly more often they may get frustrated, whereas if the user is correct significantly more frequently they may find the training boring.

Therefore, the predetermined first value above which training is made more difficult may be 95% or greater, and more preferably is 90% or greater. Equally, the predetermined second value below which training difficulty is reduced may be 70% or lower and is more preferably 80% or lower. Therefore, the success rate at which difficulty is not varied may preferably be between 70 and 95%, and more preferably is between 80 and 90%.

In preferred examples hearing training may involve a series of training sessions or rounds each involving a plurality of sequential iterations of the method. For instance, each training session may include in the range of 5 to 50 iterations, or more preferably 10 to 30 iterations. The proportion of user assessments that correctly relate to the respective information in the sequence of target audio signals across the training session may be determined at the end of each of these training sessions and the difficulty of subsequent training session(s) (i.e. rounds) may be adjusted based on this determination. As mentioned above, in some examples the number of iterations in each training session may be predetermined but this is not essential. For example, alternatively, the number of iterations in a training session may be defined by how many iterations a user is able to complete within a time limit.

In particularly preferred examples the method may involve a preliminary step of setting a baseline difficulty based on the aggregated performance of a user across a previous training session comprising a plurality of iterations. This may be performed using the predetermined first and second values as discussed above. During a subsequent training session the difficulty may be adjusted or adapted from this baseline difficulty based on the results within the training session. Therefore, the difficulty of each iteration in the training will depend on both the performance of a user in previous training sessions and on their performance in preceding iterations of the method in the ongoing (i.e. contemporaneous or current) training session.

Alternatively, after each iteration of the method the proportion of user assessments indicated by the user inputs that correctly correspond to the information defined by the respective target audio signals may be calculated for a preceding group of iterations (e.g. for the preceding 20 or 30 iterations). Therefore, the success rate for recent iterations of the method is iteratively calculated and the difficulty adjusted on a rolling basis. For example, the group of iterations on which the difficulty is based may comprise at least the 10 previous iterations, more preferably at least the 15, 20 or 30 previous iterations.

Additionally, or alternatively, the method may comprise a preliminary step of performing a standardised hearing test, and wherein based on the results of the standardised hearing test: a difficulty of the hearing training is varied; the content and/or one or more properties of the target audio signal and/or background audio signal is varied; and/or, a mode of hearing training is varied. For example, the frequency and/or volume of the target audio signal and/or background audio signal may be varied in response to the results for a user on a standardised hearing test. For instance, the frequency and volume of the target audio signal and/or background audio signal may be varied based on the results of a standardised test performed by a user. This may help ensure that the user is capable of hearing the audio signals and is able to effectively train their hearing skills. Equally, the mode of hearing training performed may be varied depending on the performance of a user at a standardised hearing test such that the user is required to perform different tasks and/or the form in which the target audio signal provides information to a user is varied. Hence the hearing training may be personalised to the specific person using the training methods discussed herein.

As examples, the standard hearing test may comprise (as previously discussed): the Amsterdam inventory for Auditory Disability and Handicap (AIADH); HearWHO (world Health Organization, 2018); a test of the highest frequency a listener can hear; a pure tone audiometry test that tests the threshold volume at which a listener can hear sounds across a range of frequencies (such as the approach defined in ISO 8253-1:2010, published by ISO, 2010-11); or any other suitable test of the hearing skills of a user. In each case, the training may be personalised to a user and therefore made more effective.

There are a wide variety of approaches by which the difficulty of hearing training may be automatically adapted. For example, increasing the difficulty of the hearing training may comprise one or more of: decreasing the volume of the target audio signal relative to the background audio signal; decreasing the quality of the target audio signal relative to the background audio signal (e.g. by applying a band pass, low pass or high pass filter to the target audio signal); increasing the similarity between the target audio signal and the background audio signal (e.g. by providing sounds in the background audio signal that are of similar frequencies to the target audio signal or are emitted from similar apparent sources relative to the user); increasing the number of sounds within the background audio signal; where the target audio signal comprises binaural audio, varying the position of the apparent source of a sound within the target audio signal relative to the user during an iteration and/or increasing the variation in positioning of one or more sounds in the target audio between sequential iterations of the method; where the background audio signal comprises binaural audio, varying the position of the apparent source(s) of one or more sounds within the background audio relative to the user during an iteration and/or increasing variation in the positioning of one or more apparent sources of respective sounds within the background audio signal relative to the user between sequential iterations of the method; increasing the complexity of the target audio signal and/or background audio signal (e.g. by using sounds that are more difficult for a user to discern or identify, including multiple of pieces of information in a target sound that a user must identify with the same user input or multiple user inputs, reducing the duration of the target audio signal, increasing the talking speed of voices in the target audio signal); applying a time limit in which a user must provide a user input; and/or increasing the visual complexity of a displayed training environment. The majority of these examples make it more difficult for a user to distinguish the target audio signal from the background audio signal and/or more difficult for the user to discern the information defined by the target audio signal. Indeed, it can be appreciated that examples such as varying the relative volumes and audio qualities of the target audio signal and background audio and increasing signal are equivalent to varying the signal to noise ratio (SNR) provided to the user. Whereas, increasing the visual complexity of a displayed training environment is more distracting for a user, requiring them to apply more concentration or attention during the training. It will also be appreciated that the difficulty of hearing training may be reduced through an opposite approach, performing the opposite of one or more the above options.

Preferably the difficulty may be incrementally adjusted after each iteration and/or training session. This may reflect the gradual or increment improvement in the hearing able of the user as they continue with the method. Each of these incremental steps of increasing or decreasing the difficulty may involve any of the actions discussed above. In particularly preferred examples the changes are arranged so as to create a gradual progression in difficulty.

In some embodiments of the method the difficulty of the training—and therefore the ability of the user to correctly identify the information in the target audio signal from the content of the background audio signal—may be quantified using the signal to noise ratio. Thus signal to noise ratio may be presented to a user or a professional as a score quantifying their performance at the hearing training and allow tracking of performance over time.

Each of the examples of how the difficulty of hearing training may be varied discussed above are common to all of the embodiments discussed above. However, the difficulty of each of the different potential modes of the invention discussed above can be varied in more specific manners.

For example, in the “foraging”/“seeking” methods discussed above the difficult number of properties by which the target audio is varied based on the relative positions of the intermediate location and target location may be reduced. As such, a user is offered less information regarding the position of the target location. Additionally, or alternatively, the position of the target location may move as discussed above. Additionally, or alternatively, the user may be required to provide a more accurate user input—i.e. the user input must be closer to the target location—to be considered to correctly correspond to the target location.

In the “identification” methods discussed above difficulty may be increased by providing multiple items of content within each target audio signal, each of which the user must correctly identify with their user input(s). For instance, where the training environment is a café, bar or restaurant the target audio signal may be “Please can I have a black coffee and a croissant” and the user may be required to provide an input or inputs relating to both the black coffee and the croissant (i.e. select visual component(s) corresponding to both products). Similarly different target audio signals and visual components used within the method may be made more similar. For example it may be more difficult for a user to distinguish a “carrot cake” from a “caraway cake” than a “carrot cake” from a “lemon cake”. Equally, the customers could provide their order from different positions relative to the user—e.g. the apparent source of the target audio signal corresponding to the customer voice could be panned from left to right or up and down relative to the user. The speed of the speaking of the customer may be increased or more obscure requests could be provided by the customer. The additional distracting sounds within the background target signal added as difficulty increases could include be one or more other customers waiting in the queue or street traffic (e.g. a car, bus or truck) passing by or other sounds from within a café. The position of the apparent of these background sounds might also vary within each iteration of the hearing training or between different iterations. A band pass filter (e.g. a filter configured to reduce the volume of certain frequencies up to 5 kHz or 3 kHz) may be applied to the target audio signal to imitate a face mask worn by a customer.

During the “matching” methods discussed above the difficulty may be increased by making target audio signals that do not correspond more similar. For instance, if a user is required to determine which target audio signals are identical, the different target audio signals may be made more similar in content (e.g. involving rhyming words, or words that differ by fewer letters or syllables), or comprise tones that are closer in pitch, reverb, duration and/or rhythm.

In further examples the difficulty of the training method may be varied by a user—i.e. the difficulty may be modified in response to receiving an input from a user. This change in difficulty may involve any of the changes discussed above. These changes in difficulty may occur between sequential iterations of the method steps or occur during a single iteration of a method. For instance, if a user is struggling to distinguish a specific target audio signal from a background audio signal during an iteration of the method they might provide an input requesting that: the target audio signal be repeated; the background audio signal be removed or reduced in volume relative to the target audio signal; a clue or hint to the target audio signal be displayed and/or that the speech within the target audio signal is displayed (e.g. using subtitles). Allowing a user to manipulate the training method in this manner may allow the hearing training to be more easily personalised to a user, and can help reduce user frustration and improve user engagement with the hearing training.

Preferably, the user device comprises a display and the method comprises displaying a training environment to a user using the display. For example the training environment may comprise an image, a video, augmented reality and/or virtual reality. Displaying a training environment to a user further increases the sensory inputs to a user during the hearing training. This increases the realism of the hearing training since users will typically experience both visual and auditory inputs in their day to day lives. Therefore, the effectiveness of the hearing training is increased. However, this is not essential and in further embodiments the hearing training may involve providing audio signals only to a user.

Displaying a training environment may allow for the complexity of the hearing training to be increased. A user may be required to relate the information defined by the target audio signal to visual components displayed within the training environment. For instance, the user may be required to select a location or item displayed within the training environment.

In equally preferred embodiments the training environment comprises virtual reality (VR) and/or augmented reality (AR) and wherein the method comprises varying the apparent source of sounds within the binaural audio of the target audio signal and/or background audio signal as a perspective from which the training environment is displayed by the user device changes.

Where the training environment comprises virtual reality (VR) the perspective from which the training environment is displayed may be varied as a user moves their head. Virtual reality systems track where the user is looking and adjusts the perspective displayed to a user through the virtual reality headset accordingly. Similarly where the training environment comprises augmented reality the perspective from which the training environment is viewed may be varied when the device displaying the augmented reality training environment (e.g. a smartphone, tablet or headset) is moved. As such, the device performs binaural synthesis, achieving accurate spatialization of sounds relative to the perspective of the user, in real-time.

The binaural audio within the target audio signal and/or background audio signal may be varied based on the change in perspective through the use of head related transfer functions (HRTF) which vary depending on the angle between the assumed user's head and the apparent source of sounds. As such, the apparent source of sounds within the binaural target audio signal and/or the background audio signal may be accurately spatialized throughout use of the AR/VR training environment. Consequently, spatialized audio may be maintained even as the perspective of a training environment displayed to a user varies. Spatialized audio is particularly realistic and accurately reflects the experiences of a user outside the VR/AR hearing training.

Preferably the target audio signal comprises one or more of: human speech; an animal call; traffic noises; a musical instrument; nature sounds; ambient noises; or synthesised sound effects. However, any suitable sound effect or recorded sound could be used within the target audio signal. As mentioned above, the target audio signal preferably comprises binaural audio, and as such the sound(s) discussed above may have an apparent position relative to the user dependent on the different signals presented to the different ears of the user.

Preferably the background audio comprises one or more of: human speech; an animal call; traffic noises; a musical instrument; weather noises; water noises; nature sounds; synthesised sounds; ambient noise; white noise; or synthesised sound effects. In further embodiments, any suitable sound effect or recorded sound could be used within the background audio signal.

More preferably the background audio comprises a plurality of sounds that at least partially overlap. Overlapping sounds in this manner, such that multiple distinct sounds are provided to a user simultaneously creates a realistic soundscape. Particularly valuable examples of background audio signals may comprise both longer, ambient sounds of relatively low volume and louder, shorter more distracting sounds. For instance, multiple human conversations can be combined to form the noise of a group of people at a café, bar or restaurant. Whilst a rainforest may be imitated by overlapping animal noises with the sounds of water dripping and foliage moving in the wind. It is particularly testing for users to distinguish the target audio signal from such complex soundscapes that comprise multiple overlapping sounds and to subsequently discern the information defined or conveyed by the target audio signal. This can improve the effectiveness of the hearing training. Indeed, as discussed above, where the background audio signal preferably comprises binaural audio, each of the overlapping sounds may have a different apparent location relative to the user. This arrangement of multiple background sounds around a user is particularly realistic and helps improve training results.

Using overlapping sounds or ambient recordings as the background audio signal (and especially sounds and recordings that are binaural) provides a highly realistic soundscape and provides improved training results in comparison to the use of a random noise signal such as white noise, pink noise or brown noise. White noise is a random signal having equal intensity at different frequencies, giving it a constant power spectral density. Pink noise or 1/f noise is a random noise signal with power spectral density which is inversely proportional to the frequency of the signal. Brown noise (also somethings called red noise) is a random noise signal with power spectral density which is inversely proportional to the frequency of the signal squared. Whilst white, pink and brown noise are consistent and easy to generate they do not reflect natural ambience and are unrealistic substitute for the ambient sounds experienced by a user in their daily life.

Preferably the target audio signals and/or background audio signals are within the human hearing range. For instance, each of the target audio signals and/or background audio signals may comprise of sound between 20 to 20,000 Hz, and more preferably between 25 Hz and 15,000 Hz, more preferably still between 100 and 10,000 Hz. Although the volume of the target audio signal may be varied relative to the background audio signal (e.g. to vary the difficulty of the training) preferably the background audio signal is quieter than the target audio signal, such that users are capable of identifying the target audio signal and determine the information conveyed by the target audio signal. For instance, the background audio signal may be at a volume of at least −6 db relative to the target audio signal and more preferably at least −12 db.

Preferably the user device is a smart user device, and wherein preferably the user device is a smart user device, and wherein preferably the user device is a smartphone, tablet, laptop, personal computer, or an AR and/or VR system. These types of personal devices are readily accessible to users, in contrast to traditional systems which are often based in laboratories. Smartphones and tablets are particularly portable and convenient for users. Whilst the use of AR and/or VR systems which include a headset may offer more complex training settings. Nevertheless any suitable user device could be used.

Preferably wherein the user device comprises a pointing device—a user interface by which allows the user to provide spatial data to the user device. For instance the user device may comprise a touchscreen, trackpad, mouse, mousepad, joystick or gamepad. However, this is not essential and any suitable input device may be used. For instance, the input device may be a microphone and the user may provide an audio input (e.g. a vocal input).

In particularly preferred examples, a display of the user device and the user interface may be combined. For instance, the user device may comprise a touchscreen. This is a particularly space efficient and intuitive means for a user to interact with the user device.

Preferably the target audio signal and background audio signal are provided to the user via headphones or an alternative audio output device connected to the audio output. The term headphones will be understood to encompass earphones, earbuds, headsets, and any other suitable form of sound output device worn on the head of a user. Headphones offer a particularly convenient means of providing the separate left and right audio channels of binaural audio directly to the corresponding ears of a user. Nevertheless in alternative embodiments the method may comprise providing the target audio signal and background audio to the user via a system of loudspeakers arranged to provide the separate right and left audio channels of binaural audio to the corresponding ears of the user.

According to a further aspect of the invention there is provided a computer implemented method of performing hearing training with a user device comprising a user interface and an audio output, the method comprising: providing a target audio signal using the audio output, the target audio signal defining information to be determined by a user; wherein the target audio signal comprises binaural audio; receiving, at the user interface, a user input corresponding to a user determination of the information defined by the target audio signal; and providing feedback to the user based on the result of the user determination.

Such methods again offer realistic and effective methods of training the hearing of a user. The use of binaural audio within a target audio signal imitates the sounds and situations a user experiences in their day-to-day lives.

Methods in accordance with this aspect of the invention may comprise any of the features discussed above with reference to the previous aspect of the invention and offer corresponding benefits, including the optional and preferable features discussed above. For instance, although a background audio signal is not essential according to this aspect of the invention, in preferred embodiments there is provided a background audio signal. This improves the realism and effectiveness of the hearing training since a user must distinguish the target audio signal from the background audio before interpreting the information conferred by the target audio signal. The background audio signal may comprise binaural audio or conventional monophonic and/or stereophonic audio.

In accordance with a further aspect of the invention there is provided a user device comprising a user interface and audio output, the user device being configured to perform a hearing training method in accordance with either of the previous aspects of the invention.

The user device may comprise any of the physical components discussed above with reference to the previous aspects of the invention and may be configured to perform any of the preferable or optional method steps discussed above. Such user devices offer corresponding benefits to the examples discussed above.

In accordance with a further aspect of the invention there is provided a non-transitory computer-readable medium storing instructions which, when read by a processor, cause a user device to perform a hearing training method according to any method discussed above.

The instructions may, when read by a processor, cause a user device to perform any of the preferable or optional method steps discussed above. Such instructions offer corresponding benefits to the examples discussed above.

### BRIEF DESCRIPTION OF DRAWINGS

Specific examples of the invention will now be discussed with reference to the following figures:

FIG. 1 schematically shows a system comprising a user device in accordance with the invention;

FIG. 2 shows a flow chart illustrating a method in accordance with the invention;

FIGS. 3a, 3b and 3c schematically show a user device performing methods in accordance with the invention;

FIG. 4 schematically shows a user device performing a method in accordance with the invention; and,

FIG. 5 schematically shows a user device performing a method in accordance with the invention.

FIG. 6 shows a flow chart illustrating a method in accordance with the invention.

### DETAILED DESCRIPTION

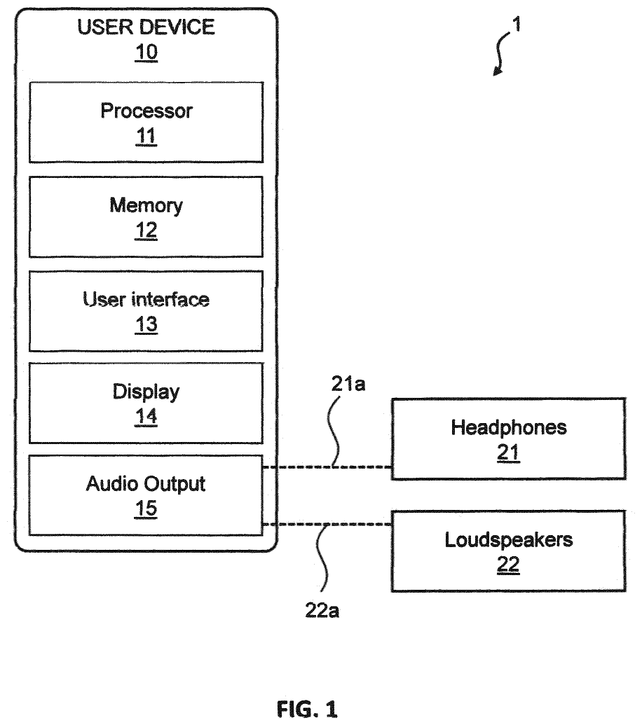

FIG. 1 shows schematically a system 1 comprising a user device 10 configured to perform a method for hearing training. The user device 10 may be a smart user device such as a smartphone, tablet, laptop or personal computer. The user device 10 comprises a processor 11, a memory 12 (i.e. a computer-readable storage medium), a user interface 13, a display 14 and audio output 15. In practice the user device may comprise further features which are not shown in this schematic drawing.

The processor 11 is configured to perform instructions recorded in the memory 12 of the user device 10. The user interface 13 is configured to receive inputs from a user (i.e. to receive user inputs). The display 14 is configured to display a training environment to the user. The user device 10 may comprise a touchscreen that provides both the user interface 13 and a display 14. Alternatively the user interface 13 and display 14 may be separate components. For instance, the user interface 13 may comprise a touchscreen, trackpad, mouse, mousepad, joystick, gamepad or any other suitable input device.

The user device 10 is configured to connect via the audio output 15 to an external audio output device such as headphones 21 or loudspeakers 22. The connections 21a, 22a between the user device 10 and the headphones 21 and/or loudspeakers 22 may be wired or wireless (e.g. via Bluetooth®, Wi-Fi®, or any other suitable alternative wireless communication protocol). The user device 10 may provide audio signals using the audio output 15 and these connections 21a, 22a which are then converted to audio (i.e. sound) by the headphones 21 or loudspeakers 22.

In particular, the user device 10 is configured to provide binaural audio to a user through the headphones 21 or loudspeakers 22. The binaural audio comprises a left and right audio channel where the difference between right and left audio channels is based on an assumed relationship between the listeners' ears (e.g. as defined by head related transfer functions (HRTF)). The user device 10 may provide binaural audio that has been binaurally recorded (i.e. recorded using a pair of microphones positioned on either side of a model head or the head of a person) or has been generated from a sample signal using head related transfer functions.

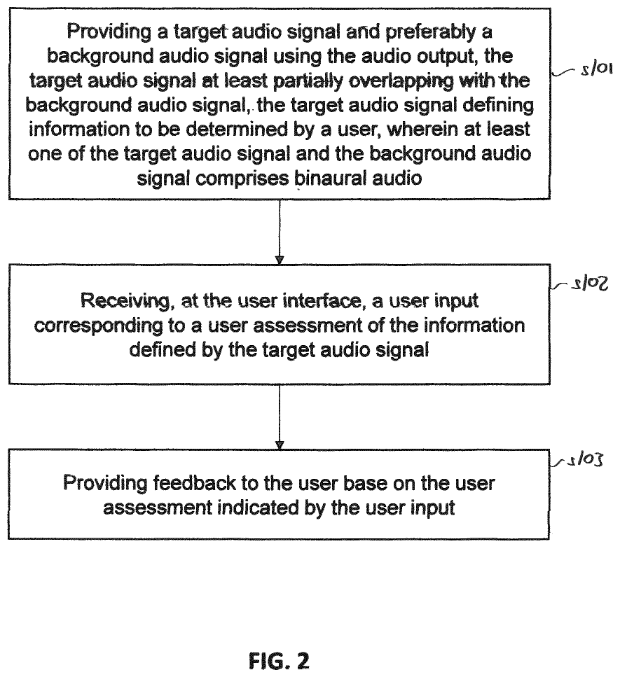

The user device 10 shown in FIG. 1 is suitable for use within the method illustrated by the flow chart of FIG. 2.

In step s101 the user device 10 provides at least a target audio signal using the audio output. Preferably the user device 10 also provides a background audio signal that at least partially overlaps with the target audio signal (e.g. such that at least some of the sound within the target audio and background audio are provided simultaneously). The target audio signal defines information to be determined by a user. At least one of the target audio signal and the background audio signal comprises binaural audio.

During training the target audio signal and any background audio signal are provided to a user by the audio output 15 of the user device 10 (e.g. via headphones 21 or loudspeakers 22). The binaural audio may be binaurally recorded and/or generated from a sample signal using head related transfer functions. Optionally, during this step the binaural audio can be adapted or dependent on the relative positioning and orientation between the head of a user and the apparent source of sound(s) within the binaural audio. The apparent source of one or more sounds within the target audio signal and/or background audio signal may be varied dependent on the position and orientation of the head of the user or the user device relative to the apparent source(s) of the sound(s). To achieve this the position and orientation of the head of the user may be tracked.

Having heard the target audio the user will attempt to discern the information defined by the target audio. Where background audio is present the user will be required to first distinguish the target audio from the background audio. The user subsequently provides a user input to the user device 10 using the user interface 13 that corresponds to their understanding or assessment of the information conveyed or conferred by the target audio. Thus, in step s102 the user device 10 receives a user input that corresponds to a user assessment of the information defined by the target audio signal.

Having received the user input s102, the user device 10 provides feedback to the user based on the user assessment indicated by the user input in step s103. Thus the user receives an evaluation of their ability to understand and interpret the audio provided from the user device 10. As such, the user is capable of training and improving their hearing.

In order to provide the feedback in step s103 the user input received at the user interface may be analysed to determine whether the user assessment corresponds to the information defined by the target audio signal. This analysis may be performed by the processor 11 of the user device 10 or any other suitable processor. If the user assessment indicated by the user input correctly corresponds to the information defined in the target audio signal (i.e. the user has correctly identified the information conveyed by the target audio) the user device 10 may receive positive feedback. Otherwise the user device 10 may provide negative feedback. The feedback may take the form of a visual indicator such as a message shown on the display 14 of the user device 10, an audio indicator such as a sound effect or verbal message provided by the audio output 15 of the user device 10 and/or any other suitable indicator such as a tactile indicator such as vibration which might be created using a vibration unit within the user device 10.

In preferred examples the method steps s101, s102, s103 are repeated iteratively to allow the user to continue training and developing their hearing skills. The difficulty of the hearing training may be progressively adjusted based on the success and/or failure of a user (i.e. a single user) in previous iterations of the method. Additionally, or alternatively the difficulty may be adjusted following a user input. Additionally or alternatively, the difficulty of the hearing training may be based on the results of a preliminary step of performing a standardised hearing test (e.g. the Amsterdam inventory, HearWHO or a test of the highest frequency a user can hear) on the user. The user device 10 may be configured to implement such a standardised hearing test via the audio output 15. However, in other examples results of a standardised hearing test may be received by the user device 10 from an external device or system. Examples of how the difficulty of different training methods may be varied or manipulated are discussed in the summary section above.

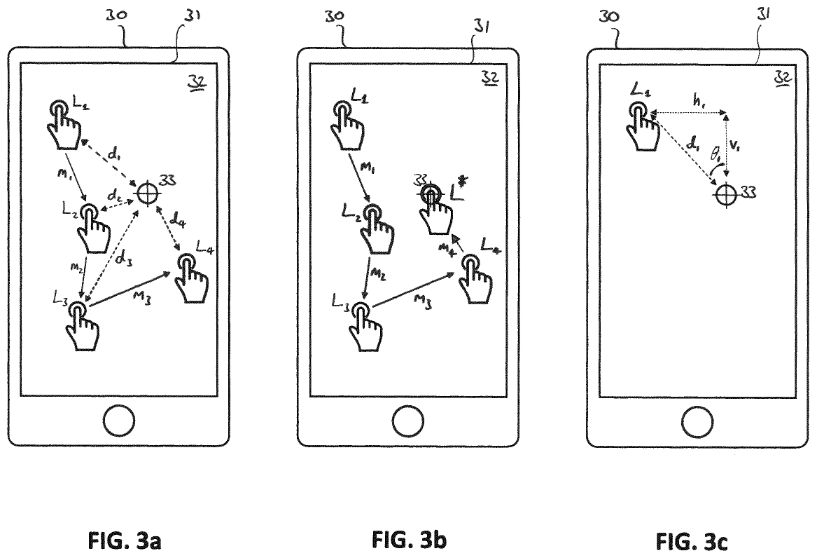

Specific examples of methods for hearing training performed with user devices 30, 40, 50 will now be discussed with reference to schematic FIGS. 3 to 5. Each of these examples incorporate the steps discussed above with reference to FIG. 2.

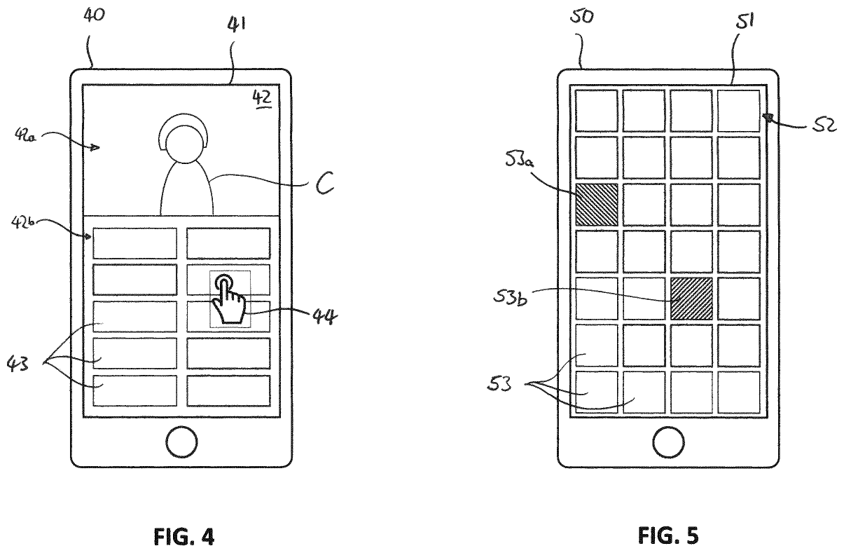

The user devices 30, 40, 50 are smartphones that comprise a touchscreen 31, 41, 51 which provides both a display and a user interface. The user devices 30, 40, 50 comprise an audio output (not shown) configured to provide target audio signals and/or background audio signals to a user (e.g. via headphones or a loudspeaker array). In each case one or both the target audio signals and the background audio signals may comprise binaural audio. In addition the user devices 30, 40, 50 may share any of the further features of the user device 10 discussed above with reference to FIGS. 1 and 2. Interactions between a user and the user devices 30, 40, 50 are shown by hand icons in FIGS. 3a to 3c and 4, and by hatched visual components in FIG. 5.

FIGS. 3a and 3b show schematically sequential steps of a “foraging” or “seeking” hearing training method performed with a user device 30. The user device 30 displays a training environment 32 to a user using the touchscreen 31. Within the training environment 32 is defined a hidden target location 33 which is not known to a user at the start of the hearing training.

FIG. 3a illustrates how the target audio signal provided by the user device 30 by the audio output of the user device 30 may be changed based on the distance d between intermediate locations L and the target location 33. Whereas, FIG. 3b shows the movement m of the user inputs through the training environment 32 as the user seeks the target location 33.

The user device 30 provides a target audio signal and preferably a background audio to the user throughout the method. The audio signals provided to the user depend on preliminary user inputs received at the touchscreen 31 from the user, these preliminary user inputs corresponding to intermediate locations L within the training environment (as shown by the hand icon in FIGS. 3a to 3c). In particular, one or more properties of the target audio signal are varied depending on the positions of the intermediate locations L relative to the hidden target location 33. As such, the target audio signal conveys information as to the location of the target location 33 within the training environment 32.

Specifically, as shown in FIG. 3a, the user device 30 receives a series of preliminary user inputs corresponding to a series intermediate locations L₁, L₂, L₃, L₄ within the training environment 32 at the touchscreen 31. For instance, the user may tap or drags their finger on the touchscreen 31 in each of the intermediate locations L₁, L₂, L₃, L₄. In other words, as the user attempts to identify the target location 33, the intermediate locations L₁, L₂, L₃, L₄ provided by each preliminary user input change, as indicated by arrows m₁, m₂, m₃, m₄.

In response to each preliminary user input, the user device 30 calculates the distance d₁, d₂, d₃, d₄ between the intermediate locations L₁, L₂, L₃, L₄ and the target location 33 and varies the properties of the target audio signal accordingly. For instance, the volume of the target audio signal may be varied (e.g. the target audio may be louder closer to the target location 33 or quieter closer to the target location 33). Additionally or alternatively, the content, pitch, duration, reverb or rhythm of the target audio may be changed. For example where the intermediate locations L are closer to the target locations 33 the target audio signal may be higher in pitch or may have a higher tempo. Additionally, or alternatively, where the target audio signal is binaural, the apparent source of the target audio signal may be varied relative to the user.

As shown in FIG. 3b, after entering a series of preliminary user inputs corresponding to the intermediate locations L₁, L₂, L₃, L₄ and hearing the resulting variations in the target audio, a user is capable of assessing or determining where the target location 33 is positioned. The user may then provide a user input corresponding to their assessment of the position L* of the target location 33 within the training environment 31 (e.g. by double tapping the touchscreen 31 of the user device 30).

Subsequently the user device 30 may determine or analyse whether the location L* indicated user input corresponds to the target location 33, and provide feedback to the user based on the result of this analysis. As shown in FIG. 3b, the location L* indicated by the user input is accurate to the target location 33 (e.g. is within a predetermined distance from the target location) and as such the user can be provided with positive feedback. However, where the user input to indicate a location far from the target location 33 the user may receive negative feedback. This feedback helps the user improve their hearing skills. The feedback may also be based on the time or number of intermediate locations required for the user to identify the target location.

A specific target audio signal that is suitable for use in this “foraging”/“seeking” method includes an animal call or sound (such as the call of a bird) which may be heard against a background audio signal soundscape of a forest which might include the separate overlapping sounds of wind against foliage, running water and further animal calls. Similarly the target audio may be the sound of a frying pan (or other piece of cooking equipment) heard against the background audio and sounds of a busy kitchen or marketplace.

It will be seen that FIGS. 3a and 3b show a series of discrete intermediate locations L₁, L₂, L₃, L₄ indicated by a series of corresponding discrete user inputs. However this is not essential and the user may input a continuous range of intermediate locations (e.g. by dragging their finger across the touchscreen 31). In this case the target audio signal provided to the user may be varied continuously.

Furthermore in FIGS. 3a and 3b the position of the target location 33 is static, but in further examples the position of the hidden target location 33 may be varied periodically or continuously.

Although as discussed above in relation to FIGS. 3a and 3b, one or more of the properties of the target audio signal can be varied dependent on the magnitude of the distance between each intermediate location L₁, L₂, L₃, L₄ and the target location 33, this is not essential. Instead, as illustrated by FIG. 3c, properties of the target audio signal may be varied dependent on the vertical distance v₁, horizontal distance h₁ and/or angle θ₁ between an intermediate location L₁ indicated by a user input and the target location 33.

In some examples a different property of the target audio may be varied based on each of these different coordinates by which the relative position of the intermediate locations L and the target location 33 can be quantified. For instance, the volume of the target audio signal relative to a background audio signal may be varied dependent on the vertical distance between the intermediate location L and the target location 33, whilst the apparent source of binaural audio within the target audio signal may be moved relative to the user depending on the horizontal distance between the intermediate location L and the target location 33. In this example a user may be required to identify a location in which the target audio is loudest and where it appears to be coming from a source directly in front of them.

FIG. 4 shows schematically a method for hearing training performed with a user device 40 (a smartphone) in which the user must “identify” the content of a target audio signal.

As in FIGS. 3a to 3c, the user device 40 displays a training environment 42 to a user using the touchscreen 40. The hearing training shown in FIG. 4 involves identifying an order placed by a customer in a café, bar or restaurant. The training environment 42 displayed by the user device 40 is divided into two sections, a customer section 42a in which customers placing orders may be displayed and a menu section 42b in which a plurality of selectable visual components 43 are displayed corresponding to different products on the menu of the café, bar or restaurant.

As a customer C is displayed by the user device 40, the user device 40 provides a target audio signal to the user (e.g. via headphones) corresponding to an order of the customer C. For instance, the target audio signal may comprise the speech “a black coffee please” or “may I have a slice of apple cake”. Consequently, the user is required to identify the products the customer C desires, and select the corresponding visual component 43. For instance, the user may provide a user input corresponding to said visual component by tapping the touchscreen of the user device 40, as indicated by hand icon 44. As such, the information defined by the target audio signal is the linguistic content of human speech within the target audio, whereas the user input is a selection of a visual component 43 that the user believes corresponds to this content of the target audio signal.

Having received the user input the user device 40 may provide feedback to the user based on the user input so as to help them improve their hearing skills. Beforehand, the user device 40 (or another device or system) may determine whether the visual component 43 indicated by the user input correctly corresponds to the content of the target audio signal. The feedback may also be based on how quickly a user provides their user input.

The user device 40 may provide a background audio such as the ambient noise of a bar, café and restaurant may be simultaneously with the target audio signal—i.e. such that the background audio signal and target audio signal overlap. As previously discussed, a particularly realistic soundscape may be created if the background audio signal comprises a plurality of overlapping sounds such as multiple human conversations, the noise of a coffee machine, cutlery and tableware and/or traffic noises. At least one of the background audio signal and target audio signal comprise binaural audio.

In the example discussed above the training environment displays customers within a café, bar or restaurant and the content of the target audio signal that a user must identify is the linguistic content of human speech (i.e. the actual words being spoken by a customer). However, this is not essential and in other examples the target audio signal and training environment may take other forms. For instance, the training environment may show a farm whilst the target audio signal comprises the noise of a farm animal. In this case the user may be required to identify the appropriate farm animal displayed by the user device 40 from its call. In such an example the background audio might include typical sounds heard on a farm.

FIG. 5 shows schematically a method for hearing training performed with a user device 50 (a smartphone) in which the user must “match” different target audio signals together.

The user device 50 displays using its touchscreen 51 a training environment 52 comprising a plurality of visual components 53 that may be selected by a user. Specifically, as shown in FIG. 5 the selectable visual components 53 take the form of tiles which may be selected by a user by tapping the touchscreen 51 on each tile. In this manner the user provides a preliminary user input to the user device 50 corresponding to a visual component 53.

For instance, the user device 50 may receive a first preliminary user input corresponding to a first visual component 53a (shown hatched in FIG. 5) and provide to the user via its audio output (not shown) a first target audio signal that corresponds to the first visual component 53a. Subsequently the user device 50 may receive a second preliminary user input corresponding to a second visual component 53b (shown hatched in FIG. 5) and provide to the user via its audio output (not shown) a second target audio signal that corresponds to the second visual component 53b. Having heard both target audio signals the user is required to assess whether the first and second target audio signals are similar and/or related—i.e. whether the target audio signals corresponding to the selected visual components 53a, 53b match. For instance, matching target audio signals may be identical and/or share similar or the same audio properties such as pitch, rhythm, duration, timbre and/or reverb. Alternatively, matching target audio signals may be conceptually linked—e.g. the first target audio signal may comprise human speech saying the word “dog”, whereas the second target audio signal may comprise the bark of a dog. Alternatively, or additionally where the target audio signals are binaural the user may be required to determine whether the target audio signals share the same apparent source—i.e. whether the target audio signals are similarly spatialized relative to the user. In this manner the information conferred to the user by the target audio signals are their relationship and/or similarity to other target audio signals.

If the user believes the target audio signals corresponding to two or more different visual components 53 are similar and/or related (e.g. the first and second visual components 53a, 53b shown in FIG. 5) they may provide a user input corresponding to said different visual components 53. For instance, the user may “double tap” each of said visual components 53 shown in the training environment and/or “drag” one of said visual components 53 to another visual component 53.

Having received the user input of visual components the user believes are linked by their corresponding target audio signals the user device 50 may provide feedback to the user based on this user input. For instance, if the user has correctly identified visual components which share target audio signals which are related and/or similar the user may receive positive feedback.

In addition to the target audio signals, the user device 50 may provide background audio signals to the user via the audio output (not shown). In these methods the user is required to distinguish the target audio from the background audio before they can begin to compare the different target audio signals. One or both of the target audio signals and the background audio signals may comprise binaural audio.

Following the discussion of FIG. 5 above it will be appreciated that a variety of similar training methods may be employed using matching techniques, and the training methods are not limited to the “tile-matching” approach shown in FIG. 5.

The methods and user devices for performing hearing training discussed above with reference to FIGS. 3 to 5 have been discussed separately. However, it will be appreciated that these techniques could form alternative training modes within a wider method. For instance, the user device may be configured to perform any of the methods discussed in relation to FIGS. 3, FIG. 4 or FIG. 5 in response to a user input, an input from an external system and/or in response to performing a standardised hearing test. For instance, a standardised hearing test may identify that a specific hearing training method would be particularly beneficial for a user and the user device may then be configured to perform said hearing training method. In this manner hearing training may be easily personalised to a user.

In addition, although the techniques discussed above in relation to FIGS. 3 to 5 each involve a smartphone (i.e. user devices 30, 40, 50) with a touchscreen display 31, 41, 51, this is not essential. In further examples alternative user devices may be used including devices and systems for the provision of AR and VR. Equally, in some examples of the invention the hearing training may not involve the display of a training environment. Instead, the training method may involve the use of a physical training environment or involve audio signals only without a visual training environment.

The components of all of the devices and systems discussed above may be connected by wired or wireless connections.

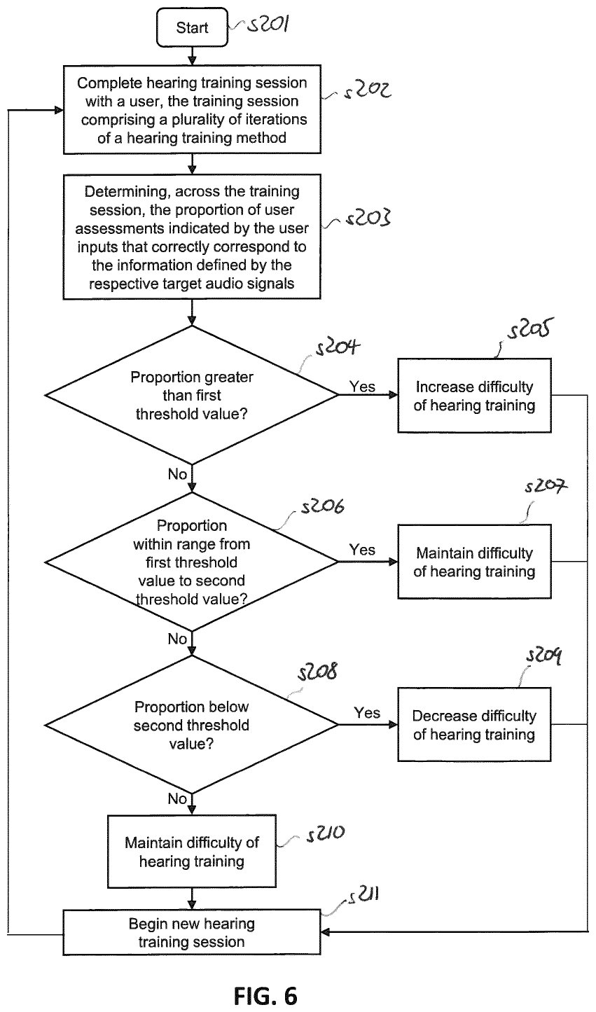

FIG. 6 shows a flow chart illustrating how the difficulty of the hearing training can be adapted to reflect the hearing ability of a user and to increase in difficulty as the hearing of a user improved. The process may be performed using the user device 10 shown in FIG. 1 and can involve any of the tasks described in reference to FIGS. 3 to 5.

In step s201 the method starts. In step s202 a hearing training session or round of hearing training comprising a plurality of iterations of a hearing training method is completed for a user (e.g. a single user). The hearing training method which is repeated in this manner may be the method described above with reference to FIG. 2. The training session may comprise at least 10 iterations of a process in which a target audio signal and background audio signal are provided to a user, a user input is received and the user input analysed to determine whether the user input to determine whether a user assessment indicated by the user input corresponds to the information defined by the target audio signal.

Subsequently, in step s203, the method involves determining, across the training session, the proportion of user assessments indicated by the user inputs that correctly correspond to the information defined by the respective target audio signals. Optionally, feedback relating to the performance of the user within the hearing training session is provided to the user based on the result of this determination. For instance, the user may be provided with a feedback score in the form of the raw percentage or a rating (e.g. a number of stars).

Based on the result of this determination in step s203 the difficulty of the training is adapted or adjusted in steps s204 to s210 as is discussed above. Following any adjustment of the difficulty of the hearing training a new hearing training session (i.e. a new round of hearing training) session may begin in step s211 and the process may repeat.

The adjustment of the difficulty begins, in step s204, with determining whether the proportion (e.g. the proportion of iterations in which the user was successful) is greater than a first threshold value. If the proportion is greater than this first threshold value the training is deemed too easy and the difficulty for future training sessions is increased in step s205. This first threshold value may be in the range of 85% to 100%, and is preferably 90%.

If the proportion is less than the first threshold value the method proceeds to step s206 where it is determined whether the proportion is within a range from the first threshold value to a second threshold value. The second threshold value may be in the range of 50 to 85% and is preferably 80%. If the proportion is in this range the training is judged to be appropriately difficult and the difficulty level for future training sessions is maintained at its existing level in step s207. Otherwise the method proceeds to step s208.

In step s208 it is determined whether the proportion is below the second threshold. If so, the hearing training is judged to be too difficult and the difficulty of future training sessions is reduced in step s209. Otherwise the difficulty is maintained at its existing level in step s210.

It should be noted that the step of determining whether the proportion is below the second threshold in step s208 is optional and redundant since it is an inherent result of the decisions in steps s204 and s206 not being met. Nevertheless actively performing the step provides redundancy and can avoid errors or issues in the calculation process.

As such, in the method shown in FIG. 6 above, involves varying the difficulty of the hearing training periodically in response to a user meeting predetermined thresholds of successful or unsuccessful user inputs over a plurality of sequential iterations of the method. Where the proportion of correct user inputs is greater than the predetermined first threshold value the difficulty of the hearing training is decreased for subsequent iterations of the hearing training process, or if the proportion of correct user inputs is less than a predetermined second threshold value the difficulty of the hearing training is increased for one or more subsequent iterations of the hearing training process.

Optionally the process discussed above is used to produce a baseline difficulty for a subsequent training session, whilst during the subsequent training session the difficulty may be varied from this baseline level based on the performance of the user during iterations of the hearing training method within the training session. As such, the difficulty is adapted based on both preceding training sessions and iterations of the method in the ongoing or current training session.

Improved training outcomes have been achieved where the first threshold value and second threshold value are 90% and 80% respectively, such that the method is continuously to maintain the success rate of the user is within the range of 80 and 90% (i.e. approximately 85%). This provides a challenge to the user that is sufficiently difficult so they do not get bored, but not so difficult that the user is frustrated. Therefore, high user engagement is achieved and users are likely to continue with hearing training and significantly improve their hearing.

It will therefore be appreciated that the method shown in FIG. 6 enables the difficulty of the hearing method may be increased or decreased incrementally to reflect changes in the hearing skills of a user. The changes in difficulty may involve any of the changes to the target audio signal, background audio signal, training environment and time scales by which a user must respond as discussed above in the summary. As such, it will be appreciated that a wide variety of progressive changes in difficulty between training sessions can be developed and predefined by those skilled in the art depending on the desired progression in difficulty. For example the difficulty may be gradually increased by incrementally decreasing the volume or quality of the target audio signal relative to the background audio signal, or by gradually increasing the number of sounds within the background audio signal and/or varying their apparent positions. Equally these changes might be applied in combination, or alternatively as required.

It is important to note that while the present invention has been described in a context of a fully functioning data processing system, those of ordinary skill in the art will appreciate that the processes of the present invention are capable of being distributed in the form of a computer readable medium of instructions and a variety of forms and that the present invention applies equally regardless of a particular type of signal bearing media actually used to carry out distribution.

Generally, any of the functionality described in this text or illustrated in the figures can be implemented using software, firmware (e.g., fixed logic circuitry), programmable or non-programmable hardware, or a combination of these implementations. The terms “component” or “function” as used herein generally represents software, firmware, hardware or a combination of these. For instance, in the case of a software implementation, the terms “component” or “function” may refer to program code that performs specified tasks when executed on a processing device or devices. The illustrated separation of components and functions into distinct units within the figures herein may reflect an actual physical grouping and allocation of such software and/or hardware, or can correspond to a conceptual allocation of different tasks performed by a single software program and/or hardware unit. Thus, the various processes described herein can be implemented on the same processor or different processors in any combination. For instance, the analysis of user inputs, the generation of feedback in response to the user inputs, the variation of the target audio signal or background audio signal and/or any of the other processes discussed above may be performed by a processor within the user device or a processor in an external device or system (e.g. a remote or cloud based system in communication with the user device).

As mentioned methods and processes described herein can be embodied as code (e.g., software code) and/or data. Such code and data can be stored on one or more computer-readable media, which may include any device or medium that can store code and/or data for use by a computer system. When a computer system reads and executes the code and/or data stored on a computer-readable medium, the computer system performs the methods and processes embodied as data structures and code stored within the computer-readable storage medium. In certain embodiments, one or more of the steps of the methods and processes described herein can be performed by a processor (e.g., a processor of a computer system or data storage system). It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that is capable of storing computer-readable information/data. Computer-readable media should not be construed or interpreted to include any propagating signals.

Although specific embodiments of the disclosure have been described, various modifications, alterations, alternative constructions, and equivalents are also encompassed within the scope of the disclosure. Embodiments of the present disclosure are not restricted to operation within certain specific data processing environments, but are free to operate within a plurality of data processing environments. Additionally, although embodiments of the present disclosure have been described using a particular series of transactions and steps, it should be apparent to those skilled in the art that the scope of the present disclosure is not limited to the described series of transactions and steps. Various features and aspects of the above-described embodiments may be used individually or jointly.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims. The modifications and variations may include any relevant combination of the disclosed features.

What is claimed is:

1. A computer implemented method of performing hearing training with a user device comprising a user interface and an audio output, the method comprising:
providing a background audio signal and a target audio signal using the audio output, the target audio signal at least partially overlapping with the background audio signal, and the target audio signal defining information to be determined by a user;
wherein one or both of the background audio signal and target audio signal comprise binaural audio;
receiving, at the user interface, a user input corresponding to a user assessment of the information defined by the target audio signal; and
providing feedback to the user based on the user assessment indicated by the user input.

2. The computer implemented method of claim 1, wherein the information defined by the target audio signal comprises:
a target location within a training environment;
the content of the target audio signal and preferably the linguistic content of speech within the target audio signal; and/or,
similarity and/or relationship to a second target audio signal.

3. The computer implemented method of claim 1, wherein the information defined by the target audio signal comprises a target location within a training environment, and wherein providing the target audio signal comprises:
receiving, at the user interface, one or more preliminary user inputs each corresponding to an intermediate location within the training environment; and
varying one or more properties of the target audio signal based on the relative positions of the intermediate location and the target location within the training environment;
wherein preferably varying one or more properties of the target audio signal comprises one or more of the following:
varying the volume of the target audio signal relative to the background audio signal;
varying the content of the target audio signal;
varying the pitch, duration, reverb and/or rhythm of the target audio signal; or,
where the target audio signal is binaural, varying the apparent source of the target audio signal relative to the user.

4. The computer implemented method of claim 1, wherein the information defined by the target audio signal corresponds to a target visual component within a plurality of different visual components within a training environment.

5. The computer implemented method of claim 1, wherein the method comprises:
providing two or more target audio signals using the audio output; and,
wherein the user input indicates whether the user believes said two or more target audio signals are similar and/or related.

6. The computer implemented method of claim 1, further comprising analysing the user input received at the user interface to determine whether the user assessment indicated by the user input corresponds to the information defined by the target audio signal.

7. The computer implemented method of claim 1, comprising iteratively repeating the method steps.

8. The computer implemented method of claim 7,
wherein either:
based on determining that the user assessment indicated by the user input corresponds to the information defined by the target audio signal, a difficulty of the hearing training is increased for subsequent iterations of the method; or,
based on determining that the user assessment indicated by the user input does not correspond to the information defined by the target audio signal, a difficulty of the hearing training is decreased for subsequent iterations of the method.

9. The computer implemented method of claim 7, wherein the difficulty of the hearing training is varied periodically in response to a user meeting a predetermined threshold of successful or unsuccessful user inputs across a plurality of sequential iterations of the method.

10. The computer implemented method of claim 7, wherein the method further comprises:
determining, across a plurality of sequential iterations of the method, the proportion of user assessments indicated by the user inputs that correctly correspond to the information defined by the respective target audio signals;
and wherein if the proportion of correct user inputs is greater than a predetermined first value the difficulty of the hearing training is decreased for one or more subsequent iterations, or if the proportion of correct user inputs is less than a predetermined second value the difficulty of the hearing training is increased for one or more subsequent iterations.

11. The computer implemented method of claim 1, comprising a preliminary step of performing a standardised hearing test; and,
wherein, based on the results of the standardised hearing test: a difficulty of the hearing training is varied;
the content and/or one or more properties of the target audio signal and/or background audio signal is varied; and/or,
a mode of hearing training is varied.

12. The computer implemented method of claim 1, wherein the user device comprises a display, and wherein the method comprises displaying a training environment to a user using the display;
and wherein preferably the training environment comprises: an image, a video, augmented reality and/or virtual reality.

13. The computer implemented method of claim 1, wherein the target audio signal comprises one or more of: human speech; an animal call; traffic noises; a musical instrument; nature sounds; ambient noises; or synthesised sound effects.

14. The computer implemented method of claim 1, wherein the background audio signal comprises one or more of: human speech; an animal call; traffic noises; a musical instrument; weather noises; water noises; nature sounds; synthesised sounds; ambient noises; white noise; or synthesised sound effects.

15. The computer implemented method of claim 1, wherein the background audio signal comprises a plurality of sounds that at least partially overlap.

16. The computer implemented method of claim 1, wherein the user device is a smart user device, and wherein preferably the user device is a smartphone, tablet, laptop, personal computer, or an AR and/or VR system.

17. A computer implemented method of performing hearing training with a user device comprising a user interface and an audio output, the method comprising:
providing a target audio signal using the audio output, the target audio signal defining information to be determined by a user;
wherein the target audio signal comprises binaural audio;
receiving, at the user interface, a user input corresponding to a user determination of the information defined by the target audio signal; and
providing feedback to the user based on the result of the user determination.

18. A user device comprising a user interface and audio output, the user device being configured to perform a hearing training method according to claim 1.

19. A non-transitory computer-readable medium storing instructions which when read by a processor cause a user device to perform a hearing training method according to claim 1.
